(19) United States
(12) Patent Application Publication
(10) Pub. No.: US 2004/0158154 A1
(43) Pub. Date: Aug. 12, 2004

(54) Portable three dimensional diagnostic ultrasound imaging methods and systems

(75) Inventors: Amin M. Hanafy, Los Altos Hills, CA (US); Anming H. Cai, San Jose, CA (US); Timothy Proulx, Santa Cruz, CA (US)

Correspondence Address: Siemens Corporation, Intellectual Property Department, 170 Wood Avenue South, Iselin, NJ, 08830, US

(73) Assignee: Siemens Medical Solutions USA, Inc.

(21) Appl. No.: 10/360,913

(22) Filed: Feb. 6, 2003

(51) Int. Cl. A61B 8/00

(52) U.S. Cl. 600/446

(57) ABSTRACT

Methods and systems are provided for three dimensional imaging with a portable diagnostic ultrasound system. Real time or four dimensional imaging with a handheld system may also be provided. A transducer array steerable in an elevation dimension is used on the portable diagnostic ultrasound system. For more rapid or simpler scanning of a volume, the transducer array is a physically steerable wobbler transducer or a transducer with a varying thickness in the elevation dimension for electronically steerable frequency dependent elevation scanning. Using a transducer array other than a fully sampled two-dimensional array may be more cost effective and may require fewer electronics within the handheld system (i.e. allow more effective miniaturization) while still providing handheld three-dimensional imaging.

### BACKGROUND

This present invention relates to diagnostic ultrasound imaging with a portable or handheld system. In particular, three dimensional imaging is provided with the handheld system.

Conventional ultrasound imaging systems typically include a hand-held transducer probe coupled by a cable to a large processing and display workstation. Limited mobility is provided by such systems. Typically, the ultrasound system is maintained in a specific location and patients are brought to the ultrasound system, but the system may be used on a wheeled cart. A more portable ultrasound system is disclosed in U.S. Pat. No. 6,312,381, the disclosure of which is incorporated herein by reference. The system shown in FIG. 11 of the '381 patent is designed to be carried as a briefcase or package by a single person, such as weighing less than 30 pounds. The system includes a large screen and a keyboard. Portability is also provided by one or more of the systems disclosed in U.S. Pat. Nos. 5,957,846, 6,251,073, 5,817,024, 6,471,651 and 6,383,139, the disclosures of which are incorporated herein by reference. Different amounts of portability are provided. For example, one system includes a hand-held scan head coupled by a cable to a portable data processor and display unit, such as a laptop computer. Other systems include a single handheld housing for the transducer, processor and a small display screen.

The handheld devices, while portable, have reduced imaging capabilities due to battery power concerns and size limitations on the amount of processing. U.S. Pat. No. 6,471,651 mentions three-dimensional (3D) imaging with a handheld device, but further specific implementation details and scanning techniques for 3D imaging are not provided.

### BRIEF SUMMARY

The present invention is defined by the following claims, and nothing in this section should be taken as a limitation on those claims. By way of introduction, the preferred embodiments described below include methods and systems for three dimensional imaging with a portable diagnostic ultrasound system. Real time or four dimensional imaging with a handheld system may also be provided.

In a first aspect, a transducer array steerable in an elevation dimension is used on the portable diagnostic ultrasound system. For more rapid or simpler scanning of a volume, the transducer array is a physically steerable wobbler transducer or a transducer with a varying thickness in the elevation dimension for electronically steerable frequency dependent elevation scanning. Using a transducer array other than a fully sampled two-dimensional array may be more cost effective and may require fewer electronics within the handheld system (i.e. allow more effective miniaturization) while still providing handheld three-dimensional imaging.

In a second aspect, a method for three dimensional imaging with a portable diagnostic ultrasound system is provided. Ultrasound energy is steered in elevation with a transducer array. A representation of a three dimensional volume is generated in response to the steering with a handheld ultrasound imaging device.

In a third aspect, a portable ultrasound system for three dimensional imaging includes a transducer array having a plurality of azimuthally spaced elements. Each of the elements has a non-uniform thickness ceramic along an elevation dimension. A first housing connects with the transducer array. The first housing is sized to be one of handheld and carried on a user. A processor within the first housing is operable to generate a representation of a three dimensional volume from information received from the transducer array. A display connects with the processor and is operable to display the representation.

In a fourth aspect, a method for three dimensional imaging with a portable diagnostic ultrasound system includes scanning in a plurality of elevationally spaced planes as a function of frequency. A representation of a three dimensional volume is generated with a handheld ultrasound imaging device as a function of the scanning of elevationally spaced planes.

Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a perspective view of one embodiment of a handheld ultrasound device;

FIG. 2 is a block diagram of one embodiment of circuitry for an ultrasound system;

FIG. 3 is a perspective view of one embodiment of a transducer array for use on a portable ultrasound system;

FIG. 4 is a representative embodiment of the use of a transducer array on a portable ultrasound system to scan in different elevation planes; and

FIG. 5 is a flow chart diagram of one embodiment of scanning for three dimensional imaging with a portable ultrasound system.

### DETAILED DESCRIPTION OF THE DRAWINGS AND PRESENTLY

### PREFERRED EMBODIMENTS

Three or four dimensional imaging on a handheld or portable ultrasound system is provided by a transducer steerable in the elevation dimension. Using the transducer steerable in the elevation dimension allows controlled scanning with a known geometry without user clamping or other control processes. A wobbler or other transducer array other than a fully sampled two-dimensional array may be more cost effective and may require fewer electronics within the handheld system (i.e. allow more effective miniaturization) while still providing handheld three-dimensional imaging. Three dimensional scanning without a two dimensional array avoids the use of complicated transducer arrays and circuitry, but a two-dimensional array may be used.

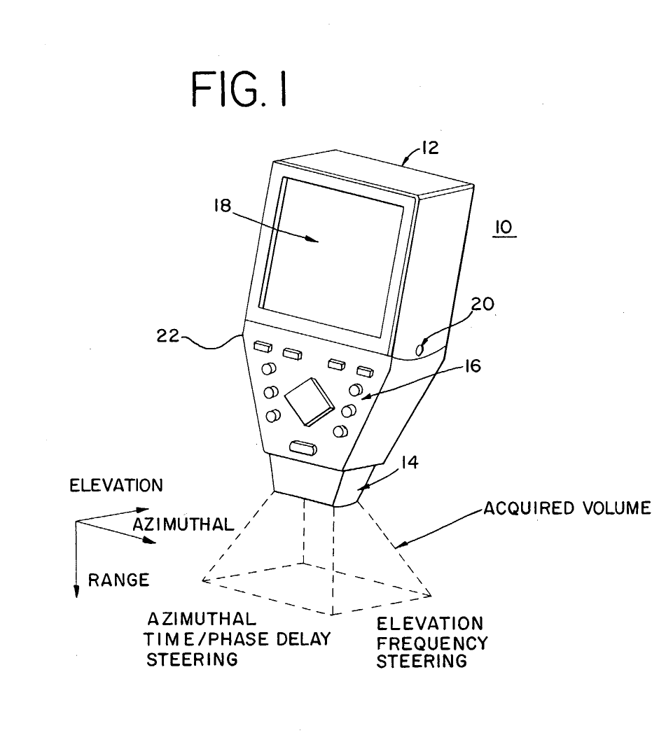

FIG. 1 shows a portable, handheld diagnostic ultrasound system 10 for three dimensional imaging. The system 10 is a handheld ultrasound image processing device 12 where an elevation steerable transducer array 14 results in a known elevation position of a scan line relative to other elevation positions. The system 10 also includes a user control 16, a display 18, a pivot connecter 20 between the display 18 and the user controls 16 and/or transducer array 14, and a housing 22. Different, fewer or additional components may be used, such as connecting the display 18 without the pivot connector 20. In other embodiments, the system 10 comprises any of the embodiments disclosed in U.S. Pat. Nos. 5,957,846, 6,251,073, 5,817,024, 6,471,651, 6,383,139, 6,312,381, ____ (Ser. No. ______ (Attorney Ref. No. 2002P15775US for an Immersive Portable Ultrasound System And Method)), U.S. Pat. No. ______ (Ser. No. ______ (Attorney Ref. No. 2002P00740US01 for a Segmented Handheld Medical Ultrasound System And Method)), and/or U.S. Pat. No. ______ (Serial No. 60/349,949 for a Medical Handheld Device), the disclosures of which are incorporated herein by reference. Different systems 10 using a single handheld unit, multiple handheld units, strap on units, portable carried units, wearable units and combinations thereof may be used.

Different levels of portability are provided. For example, FIG. 1 shows a single handheld unit. Other units with handheld components may be used. A segmented unit with one or more components strapped or connected to the users clothing also provides portability. A transducer or other ultrasound component connected with or in communication with a laptop computer or small common use display device is portable. The briefcase sized unit disclosed in U.S. Pat. No. 6,312,381 is portable (e.g. under 30 pounds and sized to be carried on or by the user), but may not be handheld.

The housing 22 comprises plastic, rubber, metal, other materials now known or later developed, or combinations thereof. The housing 22 is adapted to be portable, carried on the user, carried by the user or handheld, such as being less than 8 inches in any dimension and/or having an ergonomic shape for holding in a users hand during operation. In one embodiment, the housing 22 is sized and shaped for ergonomic use or holding by the user by having a generally round or curved circumference acting as a grip. Other shapes adapted to be held by a user's hand may be used, such as a housing with a handle to be gripped during use. In other embodiments, different shapes are used, such as a more angular, box or irregular shape with or without belt or shoulder strap attachments. For portability, the housing 22 is less than twelve, less than eight or less than six inches in one, multiple or all three spatial dimensions.

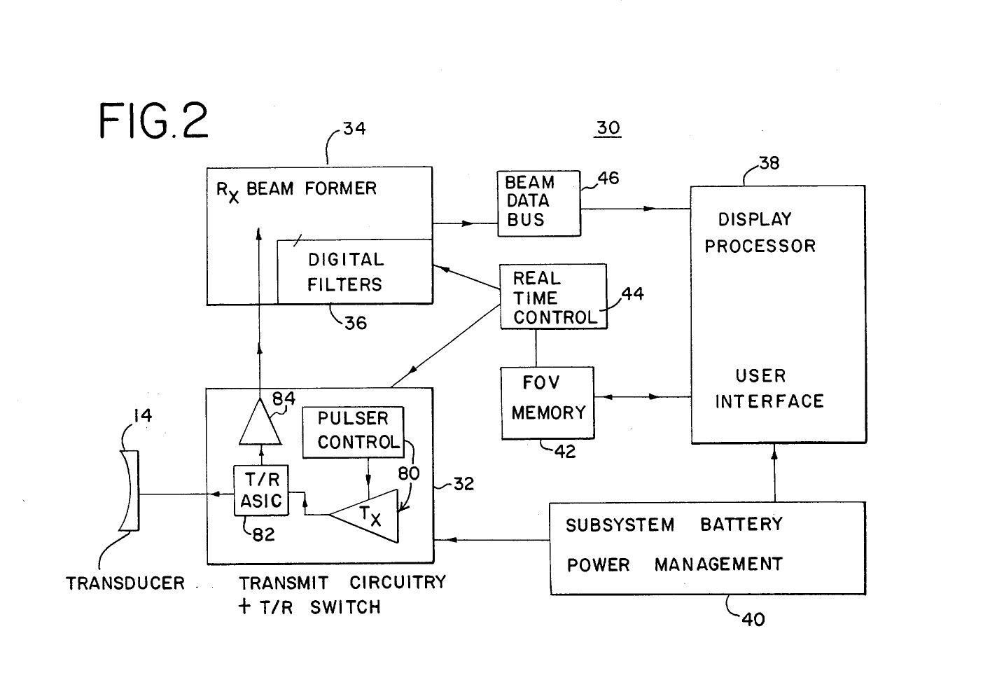

The handheld image processing ultrasound device 12 includes ultrasound circuitry 30 within or at least partly within the housing 22. FIG. 2 shows one embodiment of the ultrasound circuitry 30. The ultrasound circuitry 30 includes the transducer 14, a transmitter 32, a receive beamformer 34, filters 36, an ultrasound processor 38, a battery system 40, a memory 42, a controller 44 and a beam data bus 46. Additional, different or fewer components may be used, such as providing the transmitter and/or receive beamformers 32, 34 in a different housing, integrating the controller 44 with the ultrasound processor 38 or having multiple processors, analog circuits or digital circuits for any of the transmitter 32, the receiver 34, the ultrasound processor 38 or other components. In one embodiment, all of the digital components are integrated on a single board as high density digital electronics. Analog electronics may also be included. Alternatively, the digital and/or analog electronics are on multiple boards. In one embodiment, the ultrasound circuits 30 and/or the housing 16 are as described in the U.S. patents referenced and incorporated by reference above. While the ultrasound circuitry 30 and display 18 are within the same housing 22 in one embodiment, different components may be contained in different housings.

The transducer array 14 is steerable in an elevation dimension. Steering by the transducer array 14 is accomplished either electronically or mechanically. In one embodiment, the transducer array 14 comprises a two-dimensional array of elements for electronic steering in the elevation and azimuth dimensions. A transducer array with fewer than three elevationally spaced rows of elements is preferred. For example, the transducer array 14 comprises a wobbler transducer, such as disclosed in U.S. Pat. Nos. 4,151,834 and 4,399,822, the disclosures of which are incorporated herein by reference. A sector, linear, curved linear, 1.25D, 1.5D, 1.75D or other array of elements are mechanically wobbled or rotated along the elevation dimension to scan along different elevation positions.

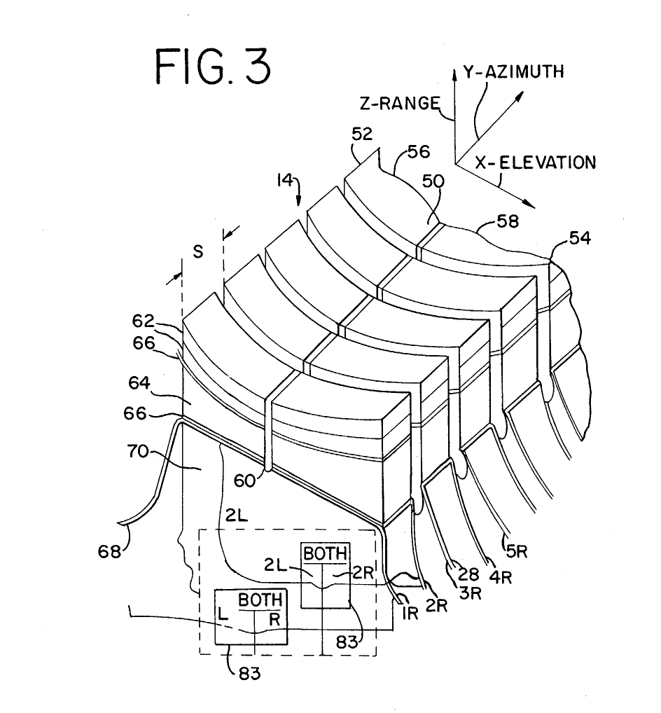

In another embodiment with fewer than three elevationally spaced rows of elements, the transducer array 14 comprises a plurality of azimuthally spaced elements in a sector, linear, curved linear or other arrangement. Each element has varying ceramic thickness or non-uniform thickness ceramic along the elevation dimension. FIG. 3 shows one embodiment of the transducer array 14 with varying ceramic thickness. Each of the elements 50 has a width in the elevation dimension extending from a first end 52 to a second end 54 and a thickness in a range dimension where the thickness of each element 50 is at a minimum at a point about midway between the first end 52 and the second end 54 and the thickness is greater than the minimum or a maximum at the first and second ends 52, 54. The thickness variation is symmetric as shown or asymmetric across the center axis. Any of various thickness variation functions may be used, such as linear, circular, parabolic, staircase (e.g. stepped), other shape functions or combinations thereof. An inverse thickness may also be used where the thickness is at a minimum at the outside edges and a maximum at the mid-point. Any of variation in the lower, upper or both surfaces may provide different thickness.

Each element 50 has a right half 56 and a left half 58 along the elevation dimension. The right half 56 is acoustically and electrically isolated from the left half 58, such as using a kerf 60 through the matching layer(s) 62, the ceramic (e.g. PZT) 64, electrodes 66, into or through the flex circuit 68 and into or not into the backing material 70. The isolation conceptually results in two elevation spaced rows of elements or a single element divided in halves 56, 58. Other transducer arrays 14 with varying ceramic thickness may be used, such as shown in Hanafy, U.S. Pat. No. 6,043,589 “Two Dimensional Transducer Array and the Method of Manufacture thereof”, in Hossack et al., U.S. Pat. No. 5,678,554 “Ultrasound Transducer for Multiple Focusing and Method for Manufacture Thereof” and in Ustuner U.S. Pat. No. 6,057,632 “Frequency and Bandwidth Controlled Ultrasound Transducer”, the disclosures of which are incorporated herein by reference.

Frequency response isolates echoes from different elevation spaced scan planes in response to the varying thickness of the transducer array 14. The varying thickness forms transmit and receive ultrasound beams along different elevation directions as a function of different frequency bands or center frequencies. Scan planes are spaced in the elevation dimension in response to the center frequency. By electronic steering in azimuth and electronic changes in frequency, beams representing a plurality of elevation spaced scan planes are obtained. The center frequency determines the elevation angle of transmission and reception based on an associated thickness along the elevation aperture. By dividing the elevation into two elements or halves 56, 58, a volume is scanned in response to different center frequencies transmitted and received by both halves 56, 58 or a single half 56, 58.

The ratio of the maximum to minimum range thickness variation of the ceramic in the elevation dimension allows for a broad band transmission response with a narrow band frequency response at a discrete frequency. In one embodiment, the ratio is 40/17 or 2.35 with a maximum thickness of 1 mm and minimum thickness of 0.43 mm, but other ratios (e.g. 2:1 to 5:1), maximum thickness and/or minimum thicknesses may be used. Intentionally mismatching the acoustic impedance of the front and/or back acoustic ports (i.e. matching layer 62 and the backing 70) with respect to the ceramic layer 64 allows for better frequency and resulting aperture control along the elevation aperture. Using a single matching layer 62 and/or tuning the matching layer thickness to be frequency dependent in correspondence with the ceramic layer 64 allows increased elevation control. Local bandwidths as narrow as 10% at −3 dB and 20% at −6 dB for a Q of 10 may be possible, but narrower or wider bandwidths may be used. In one embodiment, a low impedance or air backing 70 with an acoustic impedance of 0-2 MRayl and a single uniform or varying thickness matching layer 62 with an acoustic impedance of 3-6 MRayl are used in conjunction with the ceramic layer 64 of varying thickness with an acoustic impedance of 24-30 MRayl. In alternative embodiments, one or both of the backing and matching layers are at or closer to typical mismatch values of 4-6 MRayl and 6-10 MRayl, respectively.

During receive beamformation, an elevation aperture of about 2.4 to 2.7 wavelengths of the local or elevation scan plane frequency may be provided by the transducer array 14, but other greater or lesser aperture sizes may be used. About accounts for manufacturing tolerances. This high spatial frequency sampling rate permits elevation steering and focusing in each of multiple elevation scan planes (e.g. 3-5 elevation scan planes per half 56, 58).

In alternative embodiments, a plurality of separate elements are spaced along the elevation dimension. Two, three or more elevation spaced rows of elements are provided for elevation steering. Each element has a different thickness. For example, see U.S. Pat. No. 6,042,546, the disclosure of which is incorporated herein by reference. Different frequencies result in scanning different elevationally spaced scan lines.

In one embodiment, the transducer array 14 is sized to be small for portability, such as using more closely-spaced elements 50 adapted for higher ultrasound frequencies or using fewer elements within the array (e.g. 64 elements as opposed to 128 elements). In alternative embodiments, the transducer array 14 is larger. Any of various transducer arrays 14 now known or later developed may be used.

To avoid a high voltage supply requirements, a step-up transformer and conventional PZT elements are used, a multilayer PZT is used, a CMUT is used or combinations thereof. Any of various multilayer transducer structures may be used, such as disclosed in U.S. Pat. Nos. 5,548,564, 5,957,851, 5,945,770, 6,121,718, and ______ (Ser. No. 09/796,956), the disclosures of which are incorporated herein by reference.

In one embodiment, all of or a subset of the elements of the transducer array 14 are used for each transmit and/or receive event. Alternatively, one or more dedicated transmit elements are positioned adjacent to dedicated receive elements. By positioning transmit elements on each side of a receive array, the transmitters are capable of generating ultrasound pressure appearing to emanate from a single point in space.

The housing 22 supports and connects with the transducer array 14. For example, the housing 22 encloses the transducer array 14 and includes an acoustic window adjacent the transducer array 14. The transducer array 14 is within the housing 22 along with all or at least other portions of the ultrasound circuitry 30. In an alternative embodiment, a probe housing separate from the housing 22 for the ultrasound processor 38 is used. The transducer array 14 is within the probe housing. The transducer array 14 electrically and physically connects with the housing 22 through one or more cords or wirelessly (e.g., infrared, radio frequency or other wireless communication). Separate electrical connections may be provided for each element 50 of the transducer array 14 to the remaining ultrasound circuitry 30, but multiplexing may be used to minimize the number of cables. In other embodiments, additional ultrasound circuitry, such as the ultrasound circuitry for detecting and scan converting are provided in a separate probe housing with the transducer array 14.

With a separate probe housing or to reduce the number of analog-to-digital converters in an integrated housing 22, a multiplexer connects between the transducer array 14 and the ultrasound processor 38. The transducer array 14 may be free of further electronics or include additional electronics, such as preamplifiers, transmit and receive switches and/or portions of transmit and receive beam forming circuitry. For example, the transducer array 14 includes time division multiplexing circuitry, such as disclosed in U.S. Pat. No. ______ (application Ser. No. 10/184,461), filed Jun. 27, 2002, the disclosure of which is incorporated herein by reference. A multiplexer, amplifiers and optional time gain controls are provided for multiplexing receive channels onto a single or fewer number of cables or signal lines than elements within the transducer array 14. The multiplexer is provided between the receiver 34 and ultrasound processor 38 in other embodiments, but the circuitry 30 may be free of a multiplexer in the data path.

In one embodiment, the transducer array 14 is releasably connectable with the housing 22 and the ultrasound circuitry 30. For example, an electrical and physical connector is provided between the housing 22 and the transducer array 14. The connector is a pressure sensitive contact connector, small surface area high density connector, a PC circuit board connector or other now known or later developed connector for releasably connecting different sector, vector, linear and/or curved linear arrays to the housing 22. The releasable connection allows for different transducer arrays 14 to be connected with and supported by the housing 22. In alternative embodiments, the connection between the transducer array 14 and the housing 22 is set or otherwise permanent.

The transmitter 32 comprises a transmit beamformer 80, a transmit/receive switch 82 and receive amplifiers 84 on one or more boards or as one application specific integrated circuit. Different, fewer or additional components may be included in the transmitter 32, such as integrating the receive amplifiers 84 with the receive beamformer 34. Beamformer is used broadly to include forming a generally uniform field of energy over an entire field of view or a narrow beam representing a single scan line within the field of view.

The transmit beamformer 80 comprises one or more transmit pulsers, waveform generators, control circuits, switches, delays, timers, amplifiers, digital-to-analog converters or other now known or later developed analog or digital beamforming circuitry. In one embodiment, the transmit beamformer 32 comprises an analog application specific integrated circuit (ASIC) operating as a level shifter. For example, the ASIC includes FET devices with very low or ultra low resistance (e.g., 20 milliohms) for running on a 5 volt power supply in response to unipolar waveform signals. In alternative embodiments, a split power supply with positive and negative voltages may achieve higher acoustic power and wider receive dynamic range using bi-polar waveform signals. For each of 64 or other number of channels, two transistors drive an element during a transmit cycle, and the transmit and receive switch 82 is formed by two other transistors for isolating the receive circuitry. In another embodiment, the ASIC comprises a level shifter or amplifiers for driving the transducer array 14 with waveform signals provided to the ASIC (e.g., the transmit beamforming is performed, in part, in the ultrasound processor 38).

The transmit beamformer 80 connects with the transducer array 14 and is operable to generate a transmit waveform for ultrasound scanning, such as a narrow band, wide band, square wave, sinusoidal or other transmit waveform. A plane wave or widely dispersed transmit beam reduces power consumption with reception along multiple lines in a same or different elevation planes, reducing the number of transmit events.

In one embodiment for frequency dependent elevation focusing, the transmitter 32 generates a wideband transmit waveform including center frequencies associated with different elevation scan line positions at a same time. Any of various transmitters 32 may be used, such as a pulse wave generator that synthesizes pulses from envelope samples (see U.S. Pat. No. 5,675,554, the disclosure of which is incorporated herein by reference). In one embodiment, each transmit channel uses a same waveform with focusing delays free of apodization. The frequency of the excitation signal and the elevation halves 56, 58 used determines the elevation scan planes being scanned. In other embodiments, the transmitter 32 generates narrow band pulses for sequentially scanning one or more different elevation positions. Where the elements 50 have acoustically and electrically isolated halves 56, 58 (FIG. 3), the halves 56, 58 are sequentially or simultaneously excited using the switches 83. Different delays and apodizations are applied to different elements 50 for beamformation in the azimuth dimension.

The receive beamformer 34 comprises one or more delays, preamplifiers, amplifiers, summers, time gain control amplifiers, filters, buffers, multiplexers or other now known or later developed receiver circuitry or other circuits for generating data representing various positions within one or more elevation spaced scan planes. In one embodiment, a memory or additional parallel processing circuitry is provided for forming data representing different azimuth scan lines or elevation planes in response to a single receive event. The receive beamformer 34 connects with the filters 36. Alternatively, filtering at the receive beamformation stage is not provided. The receive beamformer 34 is operable to beamform two different elevationally spaced scan lines in response to the different frequency responses of the filters 36. One or more additional filters for isolating beamformed information at a desired frequency, such as a fundamental transmit or harmonic of the transmit frequency band, may also be provided.

The elevation scan line position filters 36 comprise at least two filters with different frequency responses connected with the transducer array 14. Digital, analog, finite frequency response, infinite frequency response, high pass, band pass, low pass, processor, application specific integrated circuits, fixed frequency response, programmable frequency response, combinations thereof or other filters may be used for filtering in the time or frequency domains. The filters 36 isolate data associated with different center frequencies based on different pass bands. A different filter response is provided for each elevation scan plane or scan line positions, such as provided for 3-10 different frequency responses. By transmitting a broadband pulse and isolating received information at multiple frequency bands in response to one transmit event, information for multiple elevation positions is obtained simultaneously, reducing the amount of time to scan a volume for real-time 3D imaging.

In one embodiment, the filters 36 and receive beamformer 34 comprise a plurality of application specific integrated circuits, such as one digital ASIC for every 16 channels of beamformation (e.g. for forming 16 scan lines in response to one transmission). Any combination of elevation and azimuth spaced scan lines may be formed in response to one transmit, such as 8 azimuthally spaced scan lines in each of two elevation scan planes or different ASICs for different elevation scan planes or filters 36. The ASICs include delays, amplifiers and summers for beamforming as well as a plurality of demodulators and base band filters for separating data into different frequency bands. The delays and amplifiers may be implemented as a coarse delay with a phase delay or fine delay adjustment performed by the amplifier. The amplifier also applies apodization. Using additional channels or memory buffers, the same data is processed to beam form data for different elevation planes or scan lines. For example, the elevation beamformer processing is time-interleaved to generate 32 narrow band receive beams associated with different center frequencies, elevation planes and/or azimuth positions. A 2 MHz (e.g. based on a 64 MHz system clock) reference frequency or bandwidth may be provided for each of the narrow-band beams. This elevation beamformation approach may be advantageous as compared to beamforming in the frequency domain since time varying apodization and delays are difficult to apply in the frequency domain. Alternatively, the received data is beamformed in the frequency domain.

In one embodiment, the receive beamformer 34 also comprises an analog ASIC for preamplification, time gain control, and multiplexing. The receive beamformer ASIC is separate from or included with the transmit beamformer ASIC. In one embodiment, the receiver 34 includes multiplexers, such as a eight 8-to-1 multiplexers, to reduce the number of analog-to-digital converters and signal interconnects. Signals from different channels are time division multiplexed with a sampling rate sufficiently high to avoid data loss (e.g. sampling rate eight times greater than the sampling rate of an individual channel). Alternatively, the receive beamformer 34 outputs signals for each channel on separate signal lines. In yet another alternative embodiment, the receive beamformer 34 includes analog or digital receive beamforming circuits. Any of the receive beamformer or other ultrasound circuitry disclosed in U.S. Provisional Patent Application No. 60/386,324, the disclosure of which is incorporated herein by reference, may be used.

The receive beamformer 34 includes analog-to-digital converters, such as a separate analog-to-digital converter for each channel or for each signal path. In one embodiment, eight 8 bit analog-to-digital converters are packaged together on one chip (e.g. one converter for each of eight multiplexed signal streams). In another embodiment, four chips each with sixteen 8 bit converters are provided (e.g. one converter for each of 64 channels). Other groupings, conversion resolutions and numbers of converters may be used. The analog-to-digital converters are spaced from the ultrasound processor 38, such as being in separate semiconductor chips. To reduce the number of signal lines and the inputs on the ultrasound processor 38, the analog-to-digital converters have a high speed serial output for each chip. Alternatively, each converter outputs to a separate signal line.

The ultrasound processor 38 comprises one or more of a digital signal processor, application specified integrated circuit, general processor, analog device, digital device, detector, transmit beamformer, receive beamformer, scan converter, filter, memory, buffer, data bus, analog devices now known or later developed, digital devices now known or later developed, and combinations thereof. In one embodiment, the ultrasound processor 38 is a single, small geometry (e.g., only digital or with minimal analog circuits) ASIC operable to detect, scan convert and video filter or process the ultrasound data communicated from the transducer array 14 or receive beamformer 34. Fewer, different or additional functions may be performed by the ultrasound processor 38, such as demultiplexing channel information, down converting, filtering, receive beamforming or controlling the system 10. The ultrasound processor 38 implements any of the various ultrasound circuitry and associated software described in the patents cited herein. Digital information is received from analog-to-digital converters separate from the ultrasound processor 38, but converters may alternatively be integrated with the ultrasound processor ASIC. Different functions may be performed by different components, such as providing multiple ultrasound processors 38 for parallel or sequential processing. The ultrasound processor 38 is operable to detect signals from the transducer array 14 in at least two elevationally spaced scan planes and generate data of a representation of a three dimensional volume from the detected signals.

The ultrasound processor 38 detects the received beamformed ultrasound data. In one embodiment, a B-mode detector is implemented to detect intensity or energy, but Doppler, flow, spectral Doppler, contrast agent or other detectors now known or later developed may alternatively or additionally be used. One or more filters, such as an axial and lateral filters, are included as part of the detector. In one embodiment, filters with fixed coefficients are used, but programmable filtering may be provided.

The ultrasound processor 38 scan converts data associated with the radial scan or acoustic pattern to generate ultrasound image data in a video format (e.g. Cartesian coordinate format). In one embodiment, a single radial scan format with possible changes in depth limits the number of operations for scan converting. Multiple scan formats and associated scan conversions may be used. Video filtering or processing may also be provided. The scan conversion is done for each of multiple elevation scan planes. The ultrasound processor 38 generates a representation of a three dimensional volume from the multiple scan planes, such as using alpha blending or other volume rendering techniques as discussed below.

In more complex embodiments, additional ultrasound functionality is provided, such as including functions and associated hardware from now known or later developed portable or larger ultrasound systems. For example, color flow, selection and use of different transducers with associated scan formats, different filtering, harmonic receiving, or providing different processes for different types of examination or applications, is provided by the ultrasound processor 38 or the ultrasound circuitry 30. In one embodiment, audio Doppler processing is also incorporated and output to one or more speakers or earphones.

The memory 42 comprises a CINE memory, a RAM, a removable memory (e.g., CD or diskette) or other now known or later developed memory. The memory 42 stores ultrasound data for three-dimensional processing, later recall or other uses. The data bus 46 transfers data in an acoustic format to the ultrasound processor 38.

The controller 44 comprises a processor, ASIC or other digital controller. The controller 44 indicates when to begin a scan and the transmitter 32 sequences through a table of relative delays to scan the patient. The controller 44 may alternatively provide the delay information to the transmitter 32. The controller 44 provides transmit frequency and receive filter frequency information to the transmitter 32 and the filters 36. The controller 44 also configures the system 10 based on user input.

The user controls 16 comprise one or more switches, sliders, buttons, sensors, a trackball, a mouse, a joy stick, a scroll wheel, a microphone (e.g. for voice control) and/or other now known or later developed input devices. In one embodiment, the user controls 20 simply include a power on/off trigger and a depth up/down rocker switch or buttons. In one example embodiment, the power on/off trigger is automatically in an “off” position and only positioned in the “on” position while held by the user to conserve power. The depth control may be used for other functions, such as increasing or decreasing an overall gain. Where a gain control input is not provided, a set gain or a software gain control function may be provided, such as disclosed in U.S. Pat. Nos. 5,579,768 and 6,398,733, the disclosures of which are incorporated herein by reference. The housing 16 is free of further user controls 20 for simplicity. Additional controls may be provided as shown in FIG. 1, such as any of various control functions provided on other portable or larger ultrasound systems. For example, a button for freezing an image, a set of buttons for menu navigation and/or dedicated mode of operation buttons (e.g. B-mode, Doppler mode, 3D mode or other modes) may be provided may be provided. In alternative embodiments, the user controls 16 and/or other components of the ultrasound device 12 are provided in a housing separate from the housing 22.

The display 18 comprises a CRT, LCD, plasma screen, a view finder (e.g., electronic displays used on camcorders or other devices to be positioned close to the eye), a personal digital assistant display, a lap top computer display, a tablet computer display, a personal computer monitor, a heads-up display, a telephone display, a cellular phone display or other now known or later developed display devices. The display 18 provides any of various resolutions, such as 320×240 pixels, lower or higher resolutions. In one embodiment, the display 18 outputs black and white information, but a color display may be used. The display 18 connects with the ultrasound processor 38 and is operable to display the 3D representation generated by the ultrasound processor 38.

Referring to FIG. 2, the battery 40 comprises a lithium, alkaline or other now known or later developed battery or battery pack. Other various sources of power may be provided for operating the ultrasound device 12, such as a plug or cord. Transmitted power, such as microwaves, may also be provided. The battery 40 connects to or within the housing 22 and electrically connects to the ultrasound circuitry 30. Any of various regulated voltages may be provided by the battery 40, such as 6, 10, 12, 20 or other voltages. In one embodiment, the battery 40 is capable of providing high current for transmitting ultrasound. A voltage divider, transformer or other device may be used to provide two or more different voltages from the battery 44, such as two voltages for operating analog and digital components. To keep the power supply as simple and as small as possible, the number of different power forms or voltages required within the handheld image processing ultrasound device 12 is reduced or kept at a minimum, such as one voltage provided for transmit and receive analog functions and a second voltage provided for analog to digital conversion and digital signal processing.

FIG. 5 shows a method for three dimensional imaging with a portable diagnostic ultrasound system. In act 90, the portable or handheld ultrasound system 10 is configured for 3D imaging. The user selects a 3D mode of imaging with the user inputs 16 or the 3D imaging is automatically configured. In response, the controller 44 instructs the transmitter 32, receive beamformer 34 and filters 36 for scanning a volume.

In act 92, ultrasound energy is steered in elevation using the transducer array 14 in the portable or handheld device 12. The elevation steering is performed with a known spacing of the elevation scan planes, such as knowing the angle of steering for a particular frequency or the position in elevation of a wobbler transducer. In one embodiment, mechanical movement of the transducer array 14 steers in elevation, such as by using a wobbler transducer. The ultrasound imaging device supports the transducer array during steering.

In another embodiment, the steering in the elevation dimension is a function of the transmit and receive frequencies. A plurality of elevationally spaced planes are scanned as a function of frequency. The line firing sequence loops through elevation slices or elevation slice combinations before azimuth lines to enable more coherent elevation beam forming. Since multiple elevation planes can be excited at the same time and are distinguishable through frequency isolation, real time three dimension imaging can be achieved. Alternatively, multiple azimuth lines are fired within a given elevation plane before azimuth lines of another elevation plane.

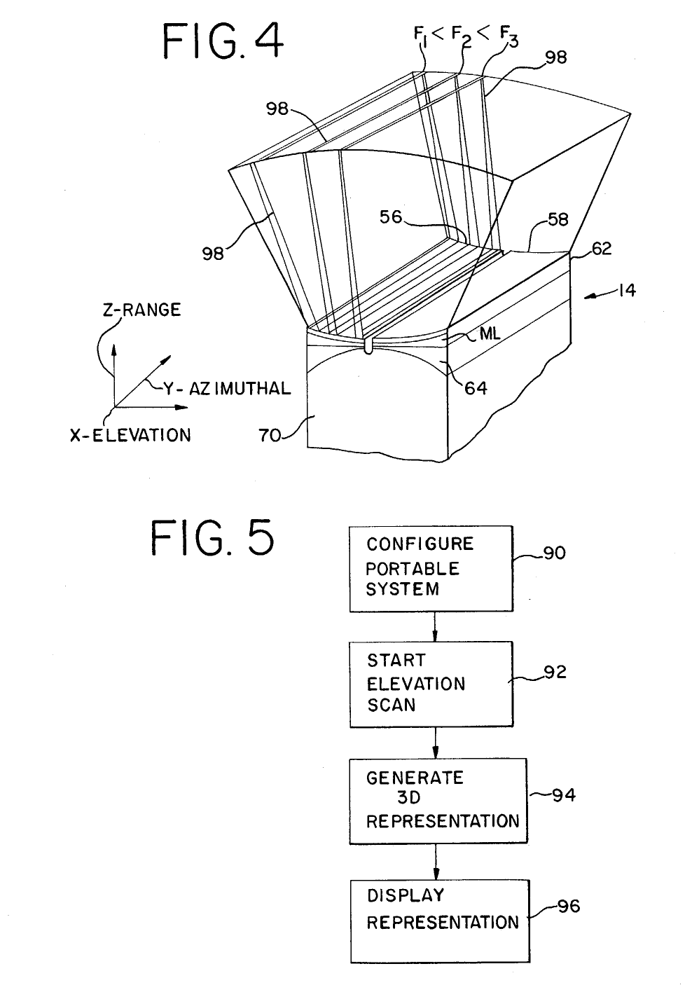

In one embodiment, excitation signals are applied to the transducer elements 50 in a first transmit event. A wide frequency band is used, such as a 2-4 MHz band of frequencies. Other wide frequency bands may be used. For example, frequency bands transmit or receive from all, most or other portions of the elevation extent of the elements 50 on one of the elevation halves 56, 58. FIG. 4 shows the transducer array 14 with different elevation spaced scan planes 98 emanating from different portions of the elevation aperture as a function of frequency. The excitation signals generate azimuth focused acoustic beams in a plurality of elevation planes (elevation fan beam).

In one embodiment, the data received in response to the wide frequency band transmit event is used to form an azimuth beam for each of the elevation planes. In an alternative embodiment, multiple transmit events are used for elevation scanning. For example, three transmit events are provided, one for one half 56, another for the other half 58 and another for both halves 56, 58. A weighted combination of data responsive to two or more of the transmit events is used for one or more of the elevation spaced planes. Data representing at least two different elevation spaced scan planes 98 is generated in response to the transmit events. Different elevation spaced scan planes 98 are associated with different center frequencies. Data for multiple elevation planes 98 are generated in response to one transmit event. Data responsive to multiple transmit events may be combined.

The data is used for elevation beamformation or for frequency and phase dependent filtering to isolate the different elevation spaced scan planes 98. For elevation beamformation, received signals are separated as a function of a plurality of frequency bands by filtering. The receive signals are separated into multiple narrower band width signals. Having a continuous element with a varying thickness in elevation allows for finer selection, natural shading or apodization, and better sensitivity than using discrete elements with different thicknesses. The separated received signals represent different elevation locations on each of the plurality of transducer elements 50. The separated received signals conceptually represent data from different elevation spaced elements. In the array 14 of FIG. 3, lower frequency filters output data corresponding to outer elements in the elevation aperture, and higher frequency filters output data corresponding to the inner elements of the elevation aperture.

The separated received signals are then beamformed in elevation for each of the different elevation spaced scan planes 98. Focusing in elevation improves elevation resolution. Since the filter banks or separated data can contribute to any given elevation scan line, range resolution may also be improved. The large and continuous nature of the element may improve the elevation beam plot since the elevation beam plot is the product of the Fourier transform of the element shape with the ideal point element elevation beam plot. Focused elevation beams are formed from the separated data representing elevation scan planes by adding the data with time delay, phase, amplitude and frequency adjustment.

Various additional methods for reducing the number of transmit events may be used to increase frame rate. Using multiple elevation beams or scan planes, intermediate elevation frames can be formed by combining or interpolating the different groups coherently or incoherently. This can be implemented either in frequency domain or time domain. Simultaneous multiple transmit beams can be fired using different frequency bands, allowing for different elevation elements to focus along different azimuth lines.

As an alternative to elevation beamformation in response to a wide-band transmit signal, the received signals are separated as a function of a plurality of frequency bands and phase responses. The beamformation is done in the frequency domain by filtering. Multiple beams are received with each beam associated with a specific frequency and phase response to match the spectrum of a point target response in a specific elevation plane and depth. Such elevation matched filters can also vary with depth along the same acoustic line to achieve dynamic focus. The filters are implemented in either frequency domain or time domain. Coded excitation signals may be used to assist in separation of information by the filters.

In yet another embodiment, one or multiple transmit narrow band ultrasound beams with different frequencies are used to sequentially excite one or a sub-set of elevation planes or scan lines at a time. Different excitation signals are applied to the plurality of transducer elements in sequential first and second transmit events. The different excitation signals have different center frequencies. Data representing at least two different elevation spaced scan planes is generated in response to the different transmit events.

In azimuth, one or more beams are formed for each transmit event. Using delays and apodization, the ultrasound energy is electronically steered in azimuth. A plane or volume wave may be transmitted for receiving energy responsive to a plurality of azimuth and/or elevation positions in response to a single transmit event. For example, a wide beam covering ⅛^{th} of the azimuth extent and all or ½ of the elevation extent of the volume is transmitted. Various elevation image-formation techniques may be used in concert with a variety of azimuthal beamformation techniques either applied to the elevation beams and/or azimuthal beams. The high frame-rate techniques disclosed in U.S. Pat. No. 6,309,356 may be used. Unfocused or weakly focused transmitted acoustic fields are used to realize high frame rates. These spatially broad transmitted fields allow the formation of image data over a substantial fraction of a frame in response to a single transmit event. The received energy is separated for elevation beamforming based on frequency and for azimuth beamforming based on delay and amplitude. The lack of lateral resolution due to the use of an unfocused transmitted field may be made up for by combining the results from a number of transmit events, each event associated with a different transmitted field angle. This technique allows the formation of a full image frame from a relatively small number of transmit events, and therefore enables very high frame-rate imaging. In combination with the frequency-dependent slice techniques described here, high frame-rate 4D imaging may be possible.

Other characteristics in addition to different center frequencies may be changed between transmit events or during receive processing. U.S. Application No. 60/386,324 describes various such characteristics. The characteristic is a function of the different center frequencies. For example, different gains are applied as a function of the center frequency. Based on frequency dependent attenuation in the tissue and/or transducer response, the transmit power (i.e. transmit gain) or receive gain is changed based on the elevation scan plane and azimuth line number or position. The gain equalizes the detected image intensity of elevation spaced scan planes so that the reconstructed 3D image has uniform appearance.

As another example characteristic that is changed in addition to center frequency, the line density or number of receive beams per transmit beams is changed. To optimize frame rate, a lower frame density may be used for low frequency outer elevation spaced scan planes. Post detection filtering can equalize the resolution across elevation spaced scan planes while preserving optimal speckle reduction for the inner elevation spaced scan planes. Phase correction changes between elevation spaced scan planes since the frequencies are changing. The phase of data responsive to one center frequency is corrected by one amount of phase shift and the phase of data responsive to a different center frequency is corrected by a different amount of phase shift. Coherent processing between elevation spaced scan planes uses cross-slice phase coherence.

In another embodiment using narrow band transmit beams as discussed above, the transmit and receive frequency bands are partially overlapped to form narrower elevation beams. As the temporal frequency bandwidth is reduced, an increasingly small elevation aperture is excited and, under the assumption that the beams are well collimated, images with an increasingly narrow elevation scan plane thickness are acquired. Instead of obtaining ever-decreasing scan plane thickness with decreasing temporal frequency bandwidth, the scan plane thickness saturates at some minimum thickness. This limitation is circumvented by transmitting and receiving with frequency bands that are centered at somewhat different frequencies, but are still at least partially overlapping. In one embodiment, this narrowing of the elevation beam is most effective when used in conjunction with a high frame-rate azimuthal beamformation technique.

The ultrasound processor 38 detects B-mode, Doppler, flow mode or other information from the beamformed data representing the scanned volume. Other data may be detected, such as harmonic B-mode information responsive to contrast agents or tissue maintained free of contrast agents during an imaging session. For harmonic imaging, a low frequency central elevation aperture pulse excitation is transmitted and higher frequency or harmonic frequency information is received, resulting in a two way elevation plane between the scan planes for the transmit and receive frequencies.

In act 94, a representation of a three dimensional volume is generated in response to the steering or as a function of scanning elevation spaced planes with a handheld ultrasound imaging device. Any of various three dimensional imaging techniques may be used, such as the harmonic or fundamental data 3D imaging disclosed in U.S. Pat. No. 5,928,151, the disclosure of which is incorporated herein by reference.

The detected data is organized as image data frames for each elevation or azimuth scan plane. The image data frames are associated with relative positional information. The two-dimensional image data frames or image planes are non-coplanar, such as two or more rotationally offset planes or two or more planes offset in elevation position. The positional information provides the relative position among the image data frames so that these frames may be subsequently assembled in a three-dimensional volume to form the desired three-dimensional reconstruction or representation. Since the elevation and azimuth position are scanned electronically or one electronically and the other mechanically, the position information relative to the transducer array 14 is known. The position information comprises three components of position (X, Y, Z) and three components of rotation (about X, Y, and Z). Other definitions of position and orientation may be used, such as two known points and one origin point on each plane. Furthermore, the position information may be assumed or measured using sensors.

The position information and the image data frames are provided to the ultrasound processor 38 and/or the memory 42. For reconstruction, the image data frames and the position information are used to generate the three dimensional representation of a volume. Information from the two-dimensional image data frames is converted to a 3D grid, such as a regularly (equal) spaced volume grid. Equal spacing allows for efficient calculations and use with low cost visualization software. The image data frame for a first plane (e.g. center plane) is inserted at a plane aligned within the volume (e.g. a center of the volume). Working outwardly from this first plane, successive image data frames are inserted into their appropriate XYZ locations, as a function of the positional information. Once all frames have been inserted, intermediate points are calculated using two or three-dimensional linear interpolation techniques or a nearest neighbor selection.

Various commercially available software is available for 3D reconstruction. For example, TomTec GmbH (Unterschleissheim, Germany) offers software specifically for 3D ultrasound. The software is capable of 3D reconstruction based on several different scan formats, such as rotations and freehand scanning. Life Imaging System Inc. (London, Ontario, Canada) also provides software for 3D ultrasound. VayTek Inc. (Fairfield, Iowa) produces rendering software for a 3D volumetric regularly spaced, orthogonal grid data. As yet another example, Advanced Visual Systems Inc. (Waltham, Mass.) offers an AVS5 software package for constructing and rendering 3D representations from the plurality of image data frames. Alternatively, the software for reconstruction of the 3D representation is written specifically for the system 10 described above. A standard language, such as C or C++, is used with custom or commercially available software tools, such as Graphics Applications Programming Interface software (e.g. OpenGL® (Silicon Graphics Inc.)). Other languages, programs, and computers may be used.

The 3D grid of 3D data samples are used for representing a three-dimensional image. Various visualization software, such as Fortner Research LLC's T3D or the software discussed above for reconstruction onto a 3D grid, and techniques may be used to present the 3D image or reconstruction on a two-dimensional display. Appropriate information is selected from the three-dimensional grid data samples or from data not on a regular 3D grid to provide a desired image. For example, cross sections can be taken in various planes, including a wide variety of planes selected by the user that do not correspond to the planes of the image data. The selected planes are interpolated from the 3D grid data samples. For 3D imaging, the 3D representation on the display 18 may be rotated, zoomed and viewed in perspective.

Various techniques for 3D imaging are possible, such as surface renderings and volume rendering displays. For surface rendering, one or more surfaces are identified by thresholding or other processes. Once the surfaces are determined, a polygon mesh is formed to represent the surface. The surface is rendered with lighting cues, such as Gouraud or Phong shading. Gouraud shading is generally simpler than Phong shading and may be accelerated with suitable hardware, but Phong shading produces a higher quality image.

Another technique for representing the 3D data samples on the display 18 is volume rendering, such as alpha blending, maximum intensity or minimum intensity projection. Based on a range of viewing angles, such as 90 or 120 degrees, and the incremental values between each viewing angle, such as 1, 3 or more degrees, a number of three dimensional projections is determined, such as 30, 90, 121 or other number. Each projection corresponds to a viewing plane that is perpendicular to the viewing angle. To minimize processing, only one, two, three or a few number of projections are determined to correspond to a current user selected viewing angle. As the image is rotated, further projections are determined. The 3D data samples at each viewing angle are summed along the lines of vision or “into” the 3D grid or viewing plane. Thus, a value for each region in a viewing plane is determined. For alpha bending, a weighting is applied to each 3D data sample. The weighting values are selected to emphasize near objects. Thus, a sense of front and back regions is created. Alpha bending allows viewing of internal objects relative to surrounding objects. Instead of alpha bending, maximum, minimum or other functions may be used. For maximum or minimum intensity projection, the maximum or minimum 3D data sample, respectively, is used instead of the summation along each line. Other viewing techniques may be used.

By minimizing the number of transmission events and/or increasing the processing, a representation of a 3D volume is repetitively generated in rapid succession or real time. For example, at least three representations based on independent scans are generated every second. Less or more rapid scanning and image generation may be used. In one embodiment, 3 to 10 elevation planes are scanned using frequency dependent focusing. Higher resolution in the elevation dimension is provided by increased processing power, decreasing the depth of each scan, increasing a number of elevation planes scanned each transmit/receive event and/or increasing a number of azimuthal beams formed by each transmit/receive event.

While the invention has been described above by reference to various embodiments, it should be understood that many changes and modifications can be made without departing from the scope of the invention. For example, the transducer array 14 and system 10 are used for 2D imaging, such as B-mode, Doppler, tissue harmonic and contrast imaging. Various coherent imaging and coherent contrast agent imaging techniques may be used in the elevation dimension or both azimuth and elevation dimension. Real time or non-real time 3D imaging are provided in a portable ultrasound system.

It is therefore intended that the foregoing detailed description be regarded as illustrative rather than limiting, and that it be understood that it is the following claims, including all equivalents, that are intended to define the spirit and scope of this invention.

I (we) claim:

1. In a handheld diagnostic ultrasound system for three dimensional imaging, the improvement comprising:
a transducer array steerable in an elevation dimension.

2. The handheld diagnostic ultrasound system of claim 1 wherein the transducer array comprises a wobbler transducer.

3. The handheld diagnostic ultrasound system of claim 1 wherein the transducer array comprises fewer than three elevationally spaced rows of elements.

4. The handheld diagnostic ultrasound system of claim 1 wherein the transducer array comprises a plurality of azimuthally spaced elements each with varying ceramic thickness along the elevation dimension.

5. The handheld diagnostic ultrasound system of claim 4 wherein each of the elements has a width in the elevation dimension extending from a first end to a second end and a thickness in a range dimension wherein the thickness of each element is at a minimum at the first end and the thickness is greater than the minimum at the second end.

6. The handheld diagnostic ultrasound system of claim 5 further comprising a second row of azimuthally spaced elements adjacent the plurality of azimuthally spaced elements, each element of the second row having a width in the elevation dimension extending from a third end to a fourth end and a thickness in a range dimension wherein the thickness is at a minimum at the third end and the thickness is greater than the minimum at the fourth end, the elements of the second row positioned adjacent the plurality of elements such that either the third end is adjacent the first end or the fourth end is adjacent the second end.

7. The handheld diagnostic ultrasound system of claim 1 further comprising:
a first housing supporting the transducer array, the housing adapted to be handheld;
one or more ultrasound processors operable to detect signals from the transducer array in at least two elevationally spaced scan planes and generate data of a representation of a three dimensional volume from the detected signals, the one or more ultrasound processors within the first housing.

8. The handheld diagnostic ultrasound system of claim 7 wherein the first housing is less than 8 inches in any dimension.

9. The handheld diagnostic ultrasound system of claim 1 further comprising:
at least two filters with different frequency responses connected with the transducer array; and
a receive beamformer connected with the at least two filters and operable to beamform two different elevationally spaced scan lines in response to the different frequency responses.

10. The handheld diagnostic ultrasound system of claim 9 further comprising:
a transmitter connected with the transducer array and operable to generate a wideband transmit waveform including center frequencies of the different frequency responses.

11. The handheld diagnostic ultrasound system of claim 1 wherein the system comprises a handheld ultrasound image processing device wherein an elevation position of a scan line is known relative to other elevation positions based on the steerable transducer array.

12. A method for three dimensional imaging with a portable diagnostic ultrasound system, the method comprising:
(a) steering ultrasound energy in elevation with a transducer array; and
(b) generating a representation of a three dimensional volume in response to the steering with a handheld ultrasound imaging device.

13. The method of claim 12 wherein (a) comprises steering as a function of frequency.

14. The method of claim 12 wherein (a) comprises steering with a wobbler transducer array.

15. The method of claim 12 wherein (a) comprises steering with the transducer array physically supported by the handheld ultrasound imaging device.

16. The method of claim 12 further comprises:
(c) repeating (b) at least three times a second.

17. The method of claim 12 wherein (a) comprises steering in elevation with a known spacing from a transducer array in a handheld housing.

18. A portable ultrasound system for three dimensional imaging, the system comprising:
a transducer array having a plurality of azimuthally spaced elements, each of the elements having a non-uniform thickness ceramic along an elevation dimension;
a first housing connected with the transducer array, the first housing sized to be one of handheld and carried on a user;
a processor within the first housing, the processor operable to generate a representation of a three dimensional volume from information received from the transducer array; and
a display connected with the processor, the display operable to display the representation.

19. The system of claim 18 further comprising:
at least two filters with different frequency responses connected with the transducer array;
a receive beamformer connected with the at least two filters and operable to beamform two different elevationally spaced scan lines in response to the different frequency responses; and
a transmitter connected with the transducer array and operable to generate a wideband transmit waveform including center frequencies of the different frequency responses.

20. The system of claim 19 wherein the at least two filters, the receive beamformer, the transmitter and the display are within the first housing, the first housing being less than eight inches in any dimension.

21. A method for three dimensional imaging with a portable diagnostic ultrasound system, the method comprising:
(a) scanning in a plurality of elevationally spaced planes as a function of frequency; and
(b) generating a representation of a three dimensional volume as a function of the scanning of elevationally spaced planes with a handheld ultrasound imaging device.

22. The handheld diagnostic ultrasound system of claim 6 wherein the third end is adjacent the first end.

23. The handheld diagnostic ultrasound system of claim 6 wherein the fourth end is adjacent the second end.

24. The system of claim 4 wherein each of the elements has a width in the elevation dimension extending from a first end to a second end and a thickness in a range dimension wherein the thickness of each element is one of a minimum and maximum at a point about midway between the first end and the second end; and
further comprising front and back acoustic ports that are mismatched in impedance with respect to the ceramic layer, the front acoustic port having an acoustic impedance of about 6 MRayl or less and the back acoustic port having an acoustic impedance of about 2 MRayl or less.

25. The system of claim 18 wherein the transducer array comprises one of: an air backing, a single matching layer, a matching layer with a thickness tuned to be frequency dependent in correspondence with the non-uniform thickness ceramic, an impedance mismatched matching layer of 6 MRayl or less with respect to the ceramic, an impedance mismatched backing of 2 MRayl or less with respect to the ceramic and combinations thereof.
